(19) United States
(12) Patent Application Publication
(10) Pub. No.: US 2026/0206184 A1
(43) Pub. Date: Jul. 16, 2026

(54) POSITIVE FRESH AIR ENCLOSURE FOR ELECTRICAL COMPONENTS

(71) Applicant: Hill Phoenix, Inc., Conyers, GA (US)

(72) Inventor: Michael Thomas Swab, Acworth, GA (US)

(21) Appl. No.: 19/016,930

(22) Filed: Jan. 10, 2025

(51) Int. Cl. H05K 7/20; G01N 33/00; H05K 5/02

(52) U.S. Cl. CPC H05K 7/20381; H05K 5/0209; H05K 7/20172; H05K 7/20209; G01N 33/0063

(57) ABSTRACT

The invention features an enclosure for electrical components. The enclosure includes an air intake and an air outlet, where a cross-sectional area of the air intake is larger than a cross-sectional area of the air outlet. The enclosure also includes a fan mounted to the enclosure and configured to blow air through the enclosure and a control circuit configured to couple with and control operation of an electrical component housed within the enclosure. The control circuit configured to, upon receiving electrical power from an external power source energize the fan, thereby, exchanging air within the enclosure and creating a positive pressure between enclosure and a surrounding environment outside the enclosure; and after a delay period, supplying power to the electrical component housed within the enclosure.

### TECHNICAL FIELD

This invention relates to enclosure for sparking electrical components.

### BACKGROUND

Refrigerated enclosures are used in commercial, institutional, and residential applications for storing and/or displaying refrigerated or frozen objects. Refrigerated enclosures may be maintained at temperatures above freezing (e.g., a refrigerator) or at temperatures below freezing (e.g., a freezer). Flammable refrigerant, e.g., R290 is used to maintain a low temperature in the refrigerated enclosures. The flammable refrigerant can create a potentially flammable environment, which has a chance to ignite with when using a spark prone electrical component. An enclosure with positive fresh air can be used to store the spark prone electrical component to prevent the flammable refrigerant from entering and contacting the spark prone electrical component during operation.

### SUMMARY

One aspect of the invention features an enclosure for electrical components, e.g., spark prone electrical components. The enclosure includes an air intake and an air outlet, a fan mounted to the enclosure and configured to blow fresh air through the enclosure, and a control circuit configured to couple with and control operation of an electrical component housed within the enclosure. The control circuit is configured to, upon receiving electrical power from an external power source, energize the fan, thereby, exchanging air within the enclosure and creating a positive pressure between enclosure and a surrounding environment outside the enclosure, and after a delay period, supplying power to the electrical component housed within the enclosure.

In some implementation, the control circuit includes a delay on make relay arranged in the control circuit to supply power to the electrical component housed within the enclosure after the delay period.

In some implementations, the electrical component is a main power relay for a refrigeration system, and where the delay on make relay is configured to close the main power relay after the delay period.

In some implementations, the delay on make relay includes a time switch configured to adjust the time delay.

In some implementations, the air intake of the enclosure is coupled to a source of fresh air.

In some implementations, the control circuit is coupled to a control power outside the enclosure through a power switch.

In some implementations, the control circuit further includes a pressure switch arranged within the control circuit to remove power from the main power relay within the housing if a pressure inside the enclosure drops below a threshold pressure.

Some implementations include a gas sensor arranged within the control circuit to remove power from the main power relay within the housing upon detection of a flammable gas inside the enclosure.

In some implementations, an air flow capacity of the air outlet is lower than an air flow capacity of the fan that is pulling air through the air intake.

Another aspect of the invention features a method of operating a spark prone electrical component within a vicinity of a potentially flammable environment, the method includes responsive to power being turning on to a control circuit for an enclosure, supplying, by the control circuit, power to a fan mounted to the enclosure, where the fan is configured to blow fresh air through the enclosure from an air intake to an air outlet. The method also includes after a delay period from the power being turned on to the control circuit, supplying power to a spark prone electrical component housed within the enclosure.

In some implementations, the air intake of the enclosure is coupled to a source of fresh air. In some implementations, an air pressure inside the enclosure is higher than an atmosphere pressure.

In some implementations, a length of the delay period is greater than a length of a time for at least two full air exchanges in the enclosure.

In some implementations, the control circuit includes a delay on make relay arranged in the control circuit, and where the method includes supplying power to the spark prone electrical component housed within the enclosure through the delay on make relay.

In some implementations, the spark prone electrical component is a main power relay for a refrigeration system, and where the method includes closing the main power relay through the delay on make relay after the delay period.

In some implementations, the delay on make relay includes a time switch, and where the method includes adjusting the length of the delay period with the time switch.

In some implementations, the enclosure further includes a pressure switch arranged within the control circuit, and where the method further includes removing power from the main power relay within the housing if a pressure inside the enclosure drops below a threshold pressure.

In some implementations, the enclosure further includes a gas sensor arranged within the control circuit, and where the method further includes removing power from the main power relay within the housing upon detection of a flammable gas inside the enclosure.

In some implementations, the control circuit is coupled to a control power outside the enclosure through a power switch.

Another aspect of the invention features a refrigerated cabinet. The refrigerated cabinet includes a refrigerated enclosure; and an enclosure configured to hold electrical components, where the enclosure is in fluid communication with air outside of the refrigerated enclosure, and where the enclosure includes an air intake and an air outlet, where a cross-sectional area of the air intake is larger than a cross-sectional area of the air outlet; a fan mounted to the enclosure and configured to blow fresh outside cabinet air through the enclosure; and a control circuit configured to couple with and control operation of an electrical component housed within the enclosure, the control circuit configured to, upon receiving electrical power from an external power source: energize the fan, thereby, exchanging air within the enclosure and creating a positive pressure between enclosure and a surrounding environment outside the enclosure; and after a delay period, supplying power to the electrical component housed within the enclosure.

In some implementations, the refrigerated enclosure includes a door frame mounted to an opening of the refrigerated cabinet, the door frame including, in cross-section:
an outer frame member of thermally conductive material and including a forward end having an outer surface arranged to be disposed outside of a refrigerated cabinet with the frame mounted, and a rearward end defining a joint; an inner frame member of thermally insulating material and including a first end retained in the joint of the outer frame member, and a second end spaced from the first end; and a sealing plate including a first edge coupled to the outer member at the rearward end of the outer member, forward of a crimp joint, a second edge supported by the second end of the inner frame member, and a sealing surface of thermally conductive material exposed to receive a door seal, where the first edge of the sealing plate is coupled to the outer member such that the sealing surface of the sealing plate and the outer surface of the forward end of the outer frame member together form a continuous heat transfer path of material more thermally conductive than the thermally insulating material of the inner frame member.

In some implementations, the refrigerated cabinet further includes a power switch mounted on the refrigerated enclosure, where the control circuit is coupled to a control power outside the enclosure through the power switch, and where the power switch is rated for a flammable environment.

The concepts described herein may provide several advantages. For example, implementations of the invention may provide a positive air pressure within the enclosure that contains electrical components. The positive air pressure enclosure is in fluid communication with air outside of the refrigerated enclosures that allows a constant fresh air exchange within the enclosure. Implementations may prevent flammable gases e.g., flammable gases such as R290, from entering the enclosure during an operation of a refrigerated enclosures. Implementations allows the use of spark prone electrical components, e.g., relays or other mechanical switches, within a vicinity of a potentially flammable environment by keep a positive fresh air pressure in the enclosure without the need to use HazLoc rated electrical components.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts a perspective view of a refrigerated enclosure having multiple doors supported by a thermal frame.

FIGS. 2A-2B depict perspective views of a refrigerated enclosure having a positive fresh air enclosure.

FIG. 3A depicts a perspective view of a positive fresh air enclosure.

FIG. 3B depicts a block diagram of a positive fresh air enclosure system according to implementations of the present disclosure.

FIG. 4 depicts a flow chart of operating a spark prone electrical component inside a positive air enclosure within a vicinity of a potentially flammable environment.

FIG. 5 depicts a circuit diagram of a positive fresh air enclosure system of FIG. 3B according to implementations of the present disclosure.

Like reference symbols in the various drawings indicate like elements.

### DETAILED DESCRIPTION

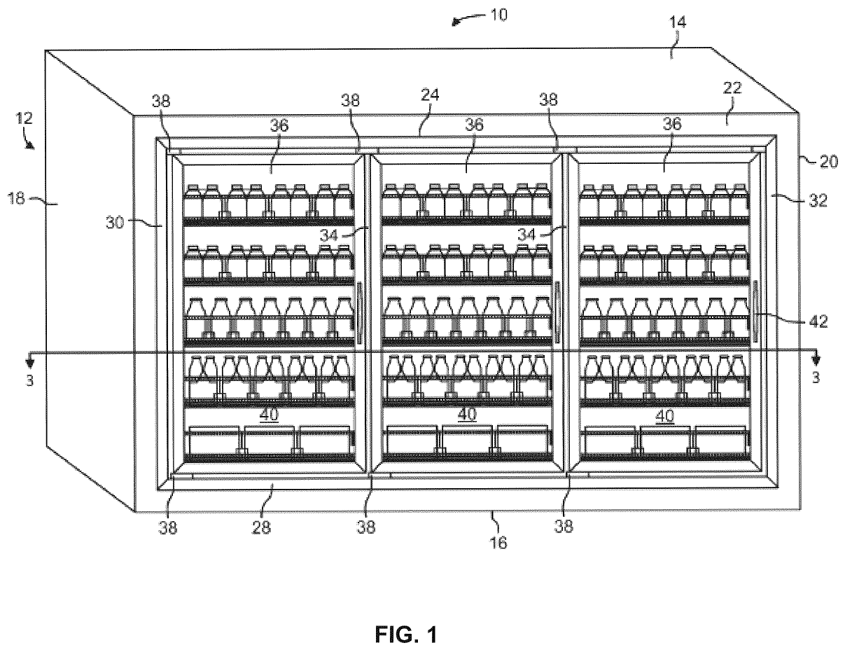

FIG. 1 shows an exemplary refrigerated enclosure 10. Refrigerated enclosure 10 may be a refrigerator, freezer, or other enclosure defining a temperature-controlled space. In some implementations, refrigerated enclosure 10 is a refrigerated display case. For example, refrigerated enclosure 10 may be a refrigerated display case or refrigerated merchandiser in grocery stores, supermarkets, convenience stores, supermarkets, convenience stores, florist shops, and/or other commercial settings to store and display temperature-sensitive consumer goods (e.g., food products and the like). Refrigerated enclosure 10 can be used to display products that must be stored at relatively low temperatures and can include shelves, glass doors, and/or glass walls to permit viewing of the products supported by the shelves. In some implementations, refrigerated enclosure 10 is a refrigerated storage unit used, for example, in warehouses, restaurants, and lounges. Refrigerated enclosure 10 can be a free standing unit or “built in” unit that forms a part of the building in which refrigerated enclosure 10 is located.

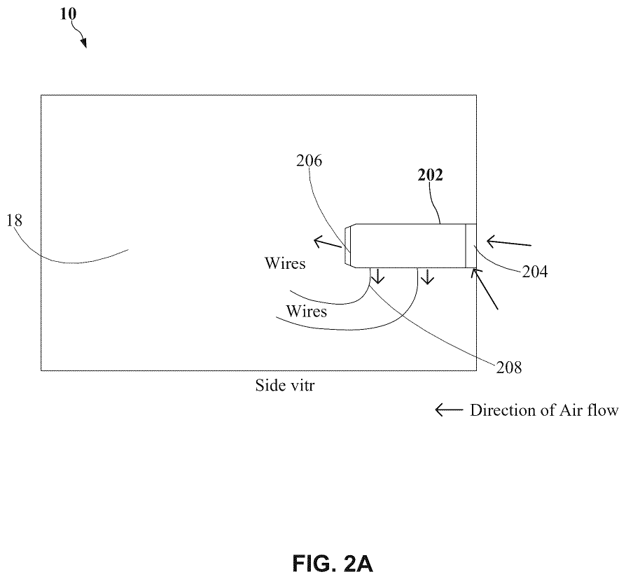

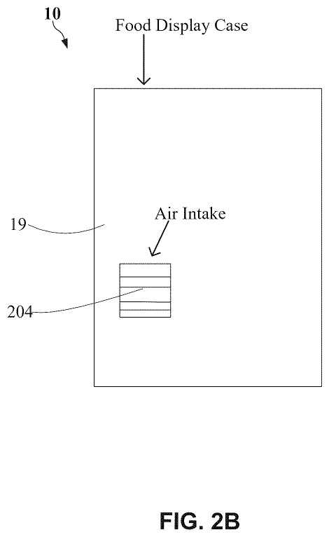

Refrigerated enclosure 10 includes a body 12. Body 12 includes a top wall 14, a bottom wall 16, a left side wall 18, a right side wall 20, a rear wall 19 (as shown in FIG. 2B), and a front portion 22 defining a temperature-controlled space. Front portion 22 includes an opening into the temperature-controlled space. Thermal frame 24 is can be mounted at least partially within the opening. Thermal frame 24 includes a plurality of perimeter frame segments (i.e., a header or top frame segment 26, a sill or bottom frame segment 28, a left side frame segment 30, and a right side frame segment 32) forming a closed shape along a perimeter of the opening. In some implementations, thermal frame 24 includes one or more mullion frame segments 34 dividing the opening into multiple smaller openings. For example, FIG. 1 depicts a three-door assembly with a pair of mullion frame segments 34 extending between top frame segment 26 and bottom frame segment 28 to divide the opening into three smaller openings. Each of the smaller openings may correspond to a separate door 36 of the three-door assembly. In other implementations, mullion frame segments 34 may be omitted. In some implementations, thermal frame 24 includes include top frame segment 26 and bottom frame segment 28 with no side frame segments 30 or 32. In such implementation, thermal frame 24 may include one or more mullion frame segments 34 depending, for example, on the size of the refrigerated enclosure in which thermal frame 24 is to be installed and the number of doors. Refrigerated enclosure 10 includes one or more doors 36 pivotally mounted on the thermal frame 24 by hinges 38. In some implementations, the doors 36 are sliding doors configured to open and close by sliding relative to the thermal frame 24. The example doors 36 illustrated in FIG. 1 include panel assemblies 40 and handles 42.

FIG. 2A depicts a side view of the refrigerated enclosure 10. A positive fresh air enclosure 202 is mounted on the side wall 18 of the refrigerated enclosure 10. The positive fresh air enclosure 202 can contain spark prone electrical components (e.g., high-voltage electrical relays, motors, heaters, mechanical timers, and power contactors, etc.), which are used to supply power to a cooling unit of the refrigerated enclosure 10. In some implementations, as shown in FIG. 2A, the positive fresh air enclosure 202 is mounted inside the refrigerated enclosure 10. Referring to FIG. 2A, the positive fresh air enclosure 202 has an air intake 204 and an air outlet 206.

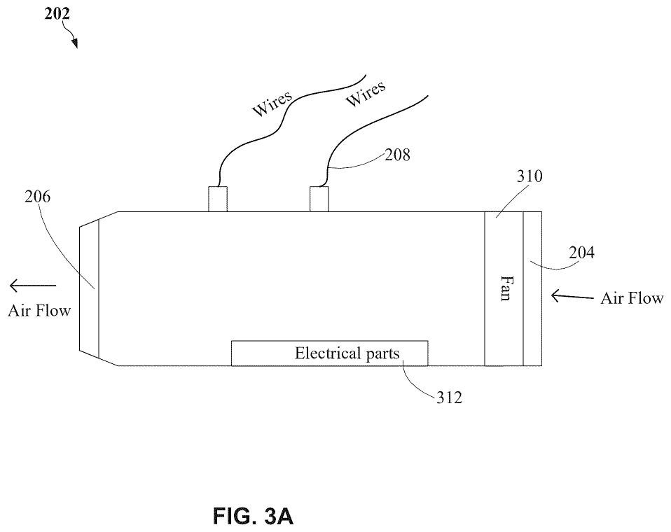

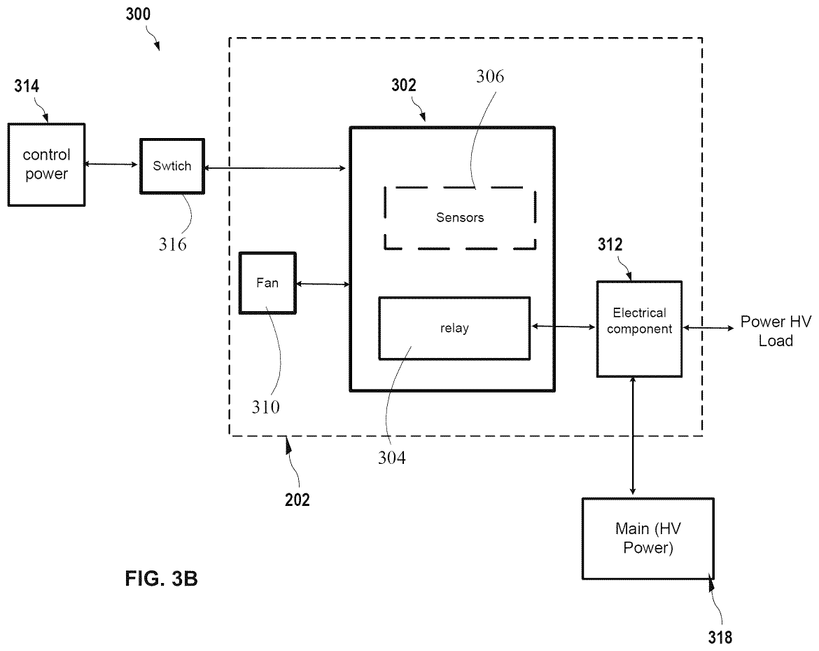

The positive fresh air enclosure 202 is configured to maintain a positive air pressure during operation. For example, a cross-sectional area of the air outlet 206 is no greater than a cross-sectional area of the air intake 204to ensure the positive air pressure inside the positive fresh air enclosure 202. In another example, a fan (e.g., the fan 310 of FIG. 3A) can be mounted inside the positive fresh air enclosure 202. The fan is configured to blow fresh air from the air intake 204 to the air outlet 206. The positive fresh air enclosure 202 is configured so that the airflow capacity of the fan 310 is greater than the total air outflow capacity of the enclosure 202 For instance, to maintain a positive pressure inside the enclosure 202, the fan 310 has an airflow capacity that is greater than the total airflow capacity of the air outlet 206 plus any airflow leakage through seams or other leakage points in the enclosure 202. In some implementations, the total airflow outlet area is the sum of the cross-sectional area of the air outlet 206 and the areas of leakage of the positive fresh air enclosure 202.

The positive fresh air enclosure 202 is in fluid communication with air outside of the refrigerated enclosure 10. For example, as shown in FIG. 2A, the air intake 204 is connected to the back wall 19 of the refrigerated enclosure 10 to ensure a fresh air supply into the positive fresh air enclosure 202. In some implementations, the positive fresh air enclosure 202 is kept at a positive pressure to prevent flammable substances from entering and to reduce the risk of flammable gas contacting the spark-prone electrical components inside the positive fresh air enclosure 202. In some implementations, the spark-prone electrical component can be a main power relay for a refrigeration system, where the main power relay is coupled to the rest of the refrigeration system through wires 208.FIG. 2C depicts a back view of the refrigerated enclosure 10. As shown in FIG. 2C, the air intake 204 of the positive fresh air enclosure 202 is mounted on the back wall 19 of the refrigerated enclosure 10. The air intake 204 can have various shapes such as a square shape (as shown in FIG. 2C), a rounded shape, and a rectangle shape. The design of the shape of the air intake 204 of the positive fresh air enclosure 202 can be changed based on the application and the size of the spark prone electrical components housed by the positive fresh air enclosure 202.

FIG. 3A depicts a perspective view of a positive fresh air enclosure 202. The positive fresh air enclosure 202 can be a part of the refrigerated enclosure 10 and is configured to house spark-prone electrical component(s) 312 during the operation of the refrigerated enclosure 10. The positive fresh air enclosure 202 has an air intake 204 and an air outlet 206 at two opposite end of the positive fresh air enclosure 202. The air intake 204 of the positive fresh air enclosure 202 is coupled to a source of fresh air. For example, the air intake 204 can be connected directly to an exterior wall of a refrigerated display cabinet to pull in air from outside the cabinet. In other examples, the air intake 204 can be coupled to an external air source through ducting or piping.

A fan 310 is mounted to the positive fresh air enclosure 202. The fan 310 is arranged to blow air through the positive fresh air enclosure 202. For example, the fan 310 can be mounted at any location inside the positive fresh air enclosure 202. For example, as shown in FIG. 3A, the fan 310 is mounted at the air intake 204 and blow the air into the positive fresh air enclosure 202 from the air intake 204. In some implementations (not shown in FIG. 3A), the fan can be mounted in the middle of the positive fresh air enclosure 202 or at the air outlet 206 of the positive fresh air enclosure 202, where the fan is configured to push the air out of the positive fresh air enclosure 202 from the air outlet 206. More generally, the fan 310 can be any device used to move air, e.g., a fan, blower, etc.

The direction of the air blown by the fan is from the air intake 204 to the air outlet 206 to ensure fresh air is coming in from the air intake 204 during the operation of the refrigerated enclosure 10. A cross-sectional area of the air intake 204 is larger than a cross-sectional area of the air outlet 206. The larger cross-sectional area of the air intake 204 can ensure a flow rate of the air coming from the air intake 204 higher than a flow rate of the air exiting from the air outlet 206 during the operation of the refrigerated enclosure 10. The difference in the flow rate of the air can help to maintain a positive pressure between the positive fresh air enclosure 202 and the surrounding environment outside the positive fresh air enclosure 202. The positive fresh air enclosure 202 is configured to house spark-prone electrical component 312. In some implementations, the spark-prone electrical component 312 can be a main power relay for a refrigeration system, where the main power relay is coupled to the remaining electrical components of the refrigeration system through wires 208.

FIG. 3B depicts a block diagram of a positive fresh air enclosure system 300 according to implementations of the present disclosure. The positive fresh air enclosure system 300 includes the positive fresh air enclosure 202 with an air intake 204 and an air outlet 206 (depicted in FIG. 3A), and a control circuit 302. The control circuit 302 of the positive fresh air enclosure 202 is coupled to control power 314. In some implementations, the control power 314 can be a wall outlet of any commercially available produces. More generally, the control power 314 can be any outlet or external power source that can supply power to the control circuit 302 of the positive fresh air enclosure 202. The control power 314 has a switch 316 that is configured to control supply of electrical power to the control circuit 302. In some implementations, an electrical power is supplied by the control power 314 to the control circuit 302 of the positive fresh air enclosure 202 upon the closing of the switch 316.

The positive fresh air enclosure 202 houses the fan 310, the control circuit 302, and the spark-prone electrical component 312. The fan 310 is coupled to the switch 316. The control circuit 302 is coupled with and configured to control operation the spark-prone electrical component 312. The control circuit 302 is configured to energize the fan 310 upon receiving electrical power from the control power 314. The fan 310 exchanges air within the positive fresh air enclosure 202 and creates a positive pressure between the positive fresh air enclosure 202 and a surrounding environment outside the positive fresh air enclosure 202.

The control circuit 302 is also configured to supply power from a main power supply 318 to the spark-prone electrical component 312 housed within the positive fresh air enclosure 202 after a delay period. The delay period allows the pressure to build within the enclosure 202 prior to supplying power to the electrical component 312. Supplying power to the electrical component 312 permits the electrical component to operate. For example, the electrical component can be an electrical power relay or contactor configured to supply electrical power to a high voltage load, e.g., a refrigeration system. For example, the electrical component 312 can be coupled between a main high-voltage (HV) power supply 318 (e.g., 120V-480V) and the high voltage load.

In some implementations, the switch 316 can be mounted on the refrigerated enclosure (e.g., the refrigerated enclosure 10 of FIG. 1A). For example, the switch 316 can be mounted on the side wall 18 or the rear wall 19 of the refrigerated enclosure 10 of FIG. 1A. The switch 316 can be a HazLoc rated electrical component that can be used in a flammable environment. For instance, the switch 316 can be turned on without causing a flame when a flammable gas (e.g., R290 refrigerant) is present.

As shown in FIG. 3B, the control circuit 302 can use a delay on make relay 304. The delay on make relay 304 is configured to supply power to the spark-prone electrical component 312 housed within the positive fresh air enclosure 202 after the delay period. In some implementations, the delay on make relay 304 is a solid-state device that can operator in a flammable environment. In some implementations, the delay on make relay 304 can include a time switch which is configured to adjust a length of the delay period. In some implementations, the spark-prone electrical component 312 is a main power relay for an electrical load, e.g., a refrigeration system. In such implementations, the electrical component 312 is electrically connected to a main (HV) power supply 318 and to the electrical load. The control circuit 302 employs the delay on make relay 304 is to provide closing power to the main power relay (electrical component 312) after the delay period.

In some implementations, as shown in FIG. 3B, the control circuit 302 can include one or more sensors 306. The control circuit 302 can include a processor or microcontroller programed to process data from the sensors 306 to confirm that conditions within the enclosure 202 are and/or remain suitable for operating the spark prone electrical component. For example, the one or more sensors 306 can be a pressure switch or pressure sensor configured to measure the pressure inside the enclosure and/or the differential pressure between the enclosure 202 and the external atmosphere. The control circuit 302 can use an input signal from the pressure switch to confirm that pressure has built to a predetermined level before operating the electrical component 312. That is, even after the delay period the control circuit 302 can further delay operating the electrical component 312 until the positive pressure is sufficiently high as measured by the pressure switch or pressure sensor. Similarly, the control circuit 302 can remove power from the spark-prone electrical component 312 if a pressure inside the positive fresh air enclosure 202 drops below a threshold pressure value, as measured by the sensor 306.

In some implementations, the one or more sensors 306 can be a gas sensor configured to detect the presence of a flammable gas inside the positive fresh air enclosure 202. In such implementations, the control circuit 302 can use input from the gas sensor to confirm whether the enclosure 202 is free of flammable gas before operating the spark-prone electrical component 312. For example, the control circuit 302 can delay operating the electrical component 312 until data from the gas sensor indicates a concentration of gas below a predetermined threshold value. As another example, the control circuit 302 can remove power from the electrical component 312 if data from the gas sensor indicates a concentration of gas above a predetermined threshold value while the electrical component 312 is already operating.

In some implementations, the control circuit 302 can include multiple types of sensors 306. For example, the sensors 306 can be a combination of different types of sensors, configured to control the spark-prone electrical component 312 under various conditions. For example, the pressure switch and the gas sensor can both be arranged inside the control circuit 302. The control circuit 302 can be configured to cut off power to the spark-prone electrical component 312 when the pressure inside the positive fresh air enclosure 202 drops below a threshold pressure value or when the gas sensor indicates detection of a flammable gas inside the positive fresh air enclosure 202. More generally, the one or more sensors 306 can be any sensors and switches used to monitor the internal conditional of the positive fresh air enclosure 202.

In some implementations, the control circuit 302 can include flow rate sensors. For example, a flow rate sensor can be installed at the air intake 204 and/or the air outlet 206 to monitor the flow of air in and out of the positive fresh air enclosure 202. The flow rate sensors can provide data to the control circuit 302 indicating proper operation of the fan 310. For example, the control circuit 302 can trigger a fault indicating improper fan operation if airflow is not detected. As another example, the control circuit 302 can monitor proper air flow through the enclosure 202 using input from one or both of an inlet airflow sensor and an outlet airflow sensor. For instance, the control circuit 302 can be configured to remove power from the spark-prone electrical component 312 if the different between the inflow air and the outflow air drops below a threshold value as indicated by the airflow sensors.

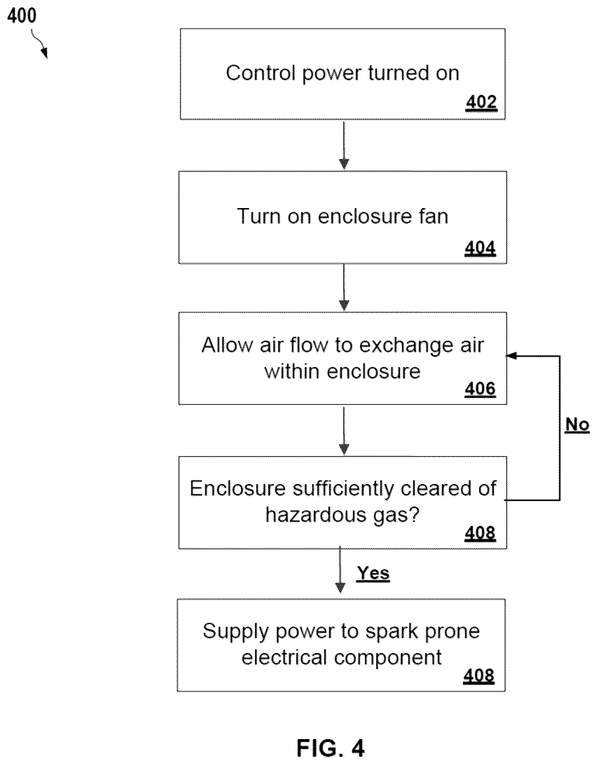

FIG. 4 depicts a flow chart of a process 400 to operate a spark prone electrical component inside a positive fresh air enclosure. The positive air enclosure can be the positive fresh air enclosure 202 as shown in FIG. 3A. The potentially flammable environment can be any flammable environment including but not limit to a refrigerated enclosure using flammable gas. For example, the flammable gas can include but not limiting to R290 (Propane) refrigerant, butane, isobutane, A2L refrigerant, and A3 refrigerant. For example, a flammable gas may be potentially flammable when exposed to a spark from the spark prone electrical component.

Process 400 can be executed by one or more computing processors including, but not limited to, the control circuit 302 described above. For example, control circuit 302 can be configured in hardware or software to perform the operations of process 400. The control circuit 302 can be provided as one or more computer executable software modules, hardware modules, or a combination thereof. For example, the control circuit 302 can be implemented as a micro controller storing blocks of software code with instructions that cause one or more processors of the controller to execute operations described herein. In addition or alternatively, the control circuit 302 can be implemented in electronic circuitry such as, e.g., programmable logic circuits, field programmable logic arrays (FPGA), or application specific integrated circuits (ASIC).

Control power is turned on (402). For example, a user can turn on control power for a system, e.g., a refrigeration system, that is controlled by a spark-prone electrical component 312 housed within a positive fresh air enclosure 202. The control power initiates operation of the positive fresh air enclosure's control circuit 302.

The control circuit 302 turns on a fan to initiate air flow through the enclosure 202 (404). For example, the control circuit 302 supplies power to fan 310 to begin exchanging the air within the enclosure 202. The fan 310 is arranged to blow air through the enclosure 202 from an air intake (e.g., the air intake 204 of FIG. 3A) to an air outlet (e.g., the air outlet 206 of FIG. 3A) that has a smaller cross-sectional area than that of the air intake. Consequently, the airflow builds a positive pressure within the enclosure 202 to prevent the entrance of potentially flammable gases from the surrounding environment.

The control circuit 302 permits the air flow to exchange air within the enclosure for a period of time (406). For example, the control circuit 302 can employ a delay timer, e.g., a delay on make relay, to permit the exchange of fresh air within the enclosure 202 for a period of time. The period of time is sufficient to expel any flammable gases from the enclosure 202 and to fill the enclosure 202 with fresh air. For example, the duration of the period of time is sufficient for at least two full air exchanges in the enclosure 202 to ensure the enclosure 202 is sufficiently clear of flammable gas. The control circuit 302 can employ one or more sensors 306 to confirm that the fan is operating, that air is flowing within the enclosure 202, that the concentration of one or more gasses is below a threshold value, that the pressure in the enclosure 202 is above a threshold value, or a combination thereof. Once either the delay period, the sensor checks, or a combination thereof indicate that the enclosure is sufficiently clear of flammable gas (408), the control circuit 302 operates the spark-prone electrical component 312. For example, the control circuit 302 supplies control power to the electrical component 312 permitting it to operate.

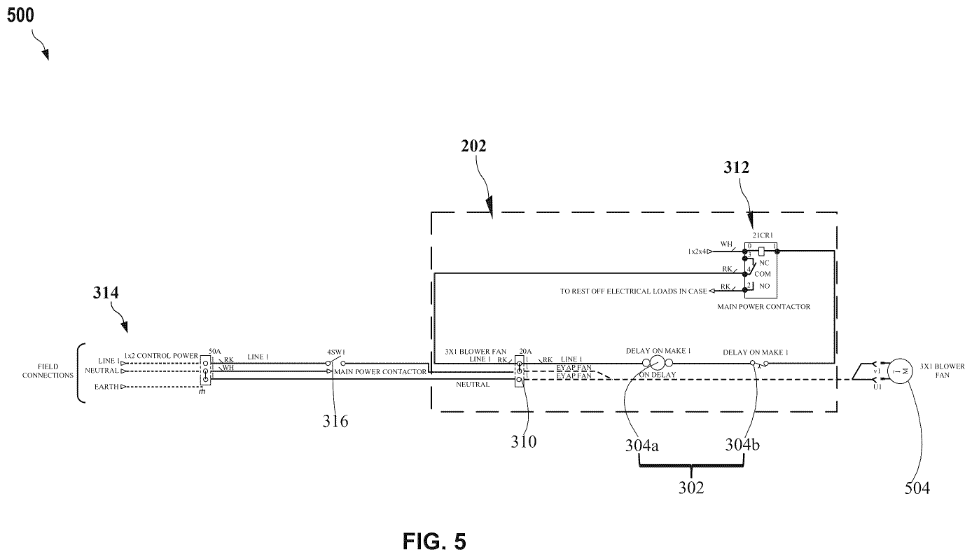

FIG. 5 depicts an example circuit 500 diagram of for controlling a positive fresh air enclosure system of FIG. 3B. The circuit diagram shown in FIG. 5 can be used to connect various components of the positive fresh air enclosure system 300 in FIG. 3B. The connection of various components shown in FIG. 5 is for illustration propose only and different circuit connections can also be used to connect the various components shown in the positive fresh air enclosure system 300 in FIG. 3B.

The example circuit 500 has two input powers. The control power 314 is coupled to the control circuit 302 of the positive fresh air enclosure 202, and the blower fan power 504 is configured to supply power directly to the fan 310. The blower fan power 504 can supply power to the fan 310 even when the control power 314 lost power to ensure a constant fresh air is blowing into the positive fresh air enclosure 202 when the switch 316 is closed.

The switch 316 is used to control the control power 314 and the positive fresh air enclosure 202. The electrical power from the control power 314 can only be applied to the control circuit 302 when the switch 316 is closed which couples the control power 314 and the control circuit 302 together. The fan 310 is enabled by the control circuit 302 once the switch 316 is closed while the power of the fan 310 is supplied by the blower fan power 504.

Upon receiving electrical power from the control power 314, the delay on make relay 304a of the control circuit 302 is enabled and start count down for a delay period. After the delay period, the delay on make relay 304a closed a delay on make switch 304b coupled to the delay on make relay 304a of the control circuit 302 to supply power to the spark-prone electrical component 312. In some implementations, the delay on make relay 304a can include a time switch configured to adjust the time delay of the delay period.

In some implementations (not shown in FIG. 5), the control circuit 302 can include one or more sensors that have a series connection with the spark-prone electrical component 312. The one or more sensors can be used to monitor the chamber conditions inside the positive fresh air enclosure 202 and remove power from the spark-prone electrical component 312 once certain conditions meet a threshold value of the one or more sensors. For example, the control circuit 302 can include a pressure switch that monitors the air pressure inside the positive fresh air enclosure 202 and removes power from the spark-prone electrical component 312 a pressure inside the positive fresh air enclosure 202 drops below a threshold pressure. The control circuit 302 can also include a gas sensor coupled to a gas sensor switch that detects flammable gases inside the positive fresh air enclosure 202 and removes power from the spark-prone electrical component 312 upon detection of a flammable gas inside the positive fresh air enclosure 202. The example of sensors shown in this disclosure are for illustration propose only, and it not intend to limit the types of sensors that can be used inside the positive fresh air enclosure 202 to monitor the conditions of the positive fresh air enclosure 202. The number of the one or more sensors inside the positive fresh air enclosure 202 is not limited to one. They can be a combination of different types of sensors, configured to control the spark-prone electrical component 312 under various conditions.

While the positive fresh air enclosure 202 has been primarily described for use with refrigeration systems, in some implementations, it can also be used to protect sparking electronics from flammable gases in other environments as well. For example, implementations of the positive fresh air enclosure 202 can be used to safely operate potentially spark causing electrical components within the vicinity of flammable gases from fueling systems, fuel handling equipment, pipelines, etc. While a number of examples have been described for illustration purposes, the foregoing description is not intended to limit the scope of the invention, which is defined by the scope of the appended claims. There are and will be other examples and modifications within the scope of the following claims. For example, the construction and arrangement of the refrigerated case with thermal door frame as shown in the various exemplary embodiments is illustrative only. Although only a few embodiments of the present inventions have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the description and advantages of the subject matter disclosed herein. For example, elements shown as integrally formed may be constructed of multiple parts or elements, the position of elements may be reversed or otherwise varied, and the nature or number of discrete elements or positions may be altered or varied. Accordingly, all such modifications are intended to be included within the scope of the present invention as defined in the appended claims. Other substitutions, modifications, changes and omissions may be made in the design, operating conditions and arrangement of the various exemplary embodiments without departing from the scope of the present inventions.

What is claimed is:

1. An enclosure for electrical components, the enclosure comprising:
an air intake and an air outlet;
a fan mounted to the enclosure and configured to blow fresh air through the enclosure; and
a control circuit configured to couple with and control operation of an electrical component housed within the enclosure, the control circuit configured to, upon receiving electrical power from an external power source:
energize the fan, thereby, exchanging air within the enclosure and creating a positive pressure between enclosure and a surrounding environment outside the enclosure; and
after a delay period, supplying power to the electrical component housed within the enclosure.

2. The enclosure of claim 1, wherein the control circuit comprises a time delay device arranged in the control circuit to supply power to the electrical component housed within the enclosure after the delay period.

3. The enclosure of claim 2, wherein the electrical component is a main power relay for a refrigeration system, and wherein the time delay device is a delay on make relay that is configured to close the main power relay after the delay period.

4. The enclosure of claim 2, wherein the delay on make relay comprises a time switch configured to adjust the delay period.

5. The enclosure of claim 1, wherein the control circuit is coupled to a control power outside the enclosure through a power switch.

6. The enclosure of claim 3, wherein the control circuit further comprises:
a pressure switch arranged within the control circuit to remove power from the main power relay within the housing if a pressure inside the enclosure drops below a threshold pressure.

7. The enclosure of claim 3, further comprises:
a gas sensor arranged within the control circuit to remove power from the main power relay within the housing upon detection of a flammable gas inside the enclosure.

8. The enclosure of claim 1, wherein an air flow capacity of the air outlet is lower than an air flow capacity of the fan that is pulling air through the air intake.

9. A method of operating a spark prone electrical component within a vicinity of a potentially flammable environment, the method comprising:
responsive to power being turning on to a control circuit for an enclosure, supplying, by the control circuit, power to a fan mounted to the enclosure, wherein the fan is configured to blow fresh air through the enclosure from an air intake to an air outlet; and
after a delay period from the power being turned on to the control circuit, supplying power to a spark prone electrical component housed within the enclosure.

10. The method of claim 9, wherein an air pressure inside the enclosure is higher than an atmosphere pressure.

11. The method of claim 10, wherein a length of the delay period is greater than a length of a time for at least two full air exchanges in the enclosure.

12. The method of claim 11, wherein the control circuit comprises a delay on make relay arranged in the control circuit, and wherein the method comprises:
supplying power to the spark prone electrical component housed within the enclosure through the delay on make relay.

13. The method of claim 12, wherein the spark prone electrical component is a main power relay for a refrigeration system, and wherein the method comprises:
closing the main power relay through the delay on make relay after the delay period.

14. The method of claim 12, wherein the delay on make relay comprises a time switch, and wherein the method comprises:
adjusting the length of the delay period with the time switch.

15. The method of claim 13, wherein the enclosure further comprises a pressure switch arranged within the control circuit, and where the method further comprises:
removing power from the main power relay t within the housing if a pressure inside the enclosure drops below a threshold pressure.

16. The method of claim 13, wherein the enclosure further comprises a gas sensor arranged within the control circuit, and wherein the method further comprises:
removing power from the main power relay within the housing upon detection of a flammable gas inside the enclosure.

17. The method of claim 9, wherein the control circuit is coupled to a control power outside the enclosure through a power switch.

18. A refrigerated cabinet comprising:
a refrigerated enclosure; and
an enclosure configured to house electrical components, wherein the enclosure is in fluid communication with air outside of the refrigerated enclosure, and wherein the enclosure comprises:
an air intake and an air outlet, wherein a cross-sectional area of the air intake is larger than a cross-sectional area of the air outlet;
a fan mounted to the enclosure and configured to blow fresh air through the enclosure; and
a control circuit configured to couple with and control operation of an electrical component housed within the enclosure, the control circuit configured to, upon receiving electrical power from an external power source:
energize the fan, thereby, exchanging air within the enclosure and creating a positive pressure between enclosure and a surrounding environment outside the enclosure; and
after a delay period, supplying power to the electrical component housed within the enclosure.

19. The refrigerated cabinet of claim 18, wherein the refrigerated enclosure comprises:
a door frame mounted to an opening of the refrigerated cabinet, the door frame comprising, in cross-section:
an outer frame member of thermally conductive material and comprising:
a forward end having an outer surface arranged to be disposed outside of a refrigerated cabinet with the frame mounted, and
a rearward end defining a joint;
an inner frame member of thermally insulating material and comprising:
a first end retained in the joint of the outer frame member, and
a second end spaced from the first end; and
a sealing plate comprising:
a first edge coupled to the outer member at the rearward end of the outer member, forward of a crimp joint,
a second edge supported by the second end of the inner frame member, and
a sealing surface of thermally conductive material exposed to receive a door seal,
wherein the first edge of the sealing plate is coupled to the outer member such that the sealing surface of the sealing plate and the outer surface of the forward end of the outer frame member together form a continuous heat transfer path of material more thermally conductive than the thermally insulating material of the inner frame member.

20. The refrigerated cabinet of claim 18, wherein the refrigerated cabinet further comprises a power switch mounted on the refrigerated enclosure, wherein the control circuit is coupled to a control power outside the enclosure through the power switch, and wherein the power switch is rated for a flammable environment.
